(19) United States
(12) Patent Application Publication
(10) Pub. No.: US 2016/0193045 A1
(43) Pub. Date: Jul. 7, 2016

(54) SELF-ASSEMBLING PERCUTANEOUSLY IMPLANTABLE HEART VALVE

(71) Applicant: MAYO FOUNDATION FOR MEDICAL EDUCATION AND RESEARCH, Rochester, MN (US)

(72) Inventor: Peter M. Pollak, Atlantic Beach, FL (US)

(21) Appl. No.: 14/912,450

(22) PCT Filed: Aug. 26, 2014
(86) PCT No.: PCT/US14/52616

§ 371 Date: Feb. 17, 2016

Related U.S. Application Data
(60) Provisional application No. 61/871,507, filed on Aug. 29, 2013.

(51) Int. Cl. A61F 2/24

(52) U.S. Cl. CPC A61F 2/2427; A61F 2/2403; A61F 2/2409; A61F 2/2445; A61F 2230/0069

(57) ABSTRACT

Devices and methods for the treatment of heart conditions. For example, a prosthetic heart valve and a transcatheter heart valve replacement method. The prosthetic heart valve can be configured into a low-profile configuration for containment within a small diameter delivery sheath.

### CROSS-REFERENCE TO RELATED APPLICATIONS

This application claims the benefit of U.S. Provisional Application Ser. No. 61/871,507, filed Aug. 29, 2013. The disclosure of the prior application is considered part of (and is incorporated by reference in) the disclosure of this application.

### BACKGROUND

1. Technical Field

This document relates to devices and methods for the treatment of heart conditions. For example, this document relates to a prosthetic heart valve and a transcatheter heart valve replacement method.

2. Background Information

Cardiac valvular stenosis is a condition in which the heart's valves are narrowed (stenotic). With valvular stenosis, the tissues forming the valve leaflets become stiffer, narrowing the valve opening, and reducing the amount of blood that can flow through it. If the stenosis is mild, the overall cardiac output remains normal. However, when the valves can become severely stenotic, that can lead to a reduction in cardiac output and impairment of heart function.

Aortic valve stenosis affects approximately 5% of all people over age 75 years. Aortic valve stenosis occurs when the heart's aortic valve narrows. When the aortic valve is so obstructed, the heart has to work harder to pump blood to the body. Eventually, this extra work limits the amount of blood the heart can pump, and may weaken the heart muscle. The left atrium may enlarge as pressure builds up, and blood and fluid may then collect in the lung tissue (pulmonary edema), making it hard to breathe. Medications can ease symptoms of mild to moderate aortic valve stenosis. However, the only way to treat severe aortic valve stenosis is by surgery to replace the valve.

Therapies to repair or replace the aortic valve include balloon valvuloplasty (valvotomy), surgical aortic valve replacement, and transcatheter aortic valve replacement (TAVR). TAVR involves replacing the aortic valve with a prosthetic valve that is delivered via the femoral artery (transfemoral) or the left ventricular apex of the heart (transapical). TAVR is sometimes referred to as transcatheter aortic valve implantation (TAVI).

The two most common complications following TAVR are vascular complications and stroke. Both complications are thought to be related to the size of the delivery sheath, and felt to be less prevalent with smaller delivery sheaths. The major limitation of reducing the size of the delivery sheath is the size of the compressed prosthetic aortic valve within the sheath.

### SUMMARY

This document provides devices and methods for the treatment of heart conditions. For example, this document provides a prosthetic heart valve and a transcatheter heart valve replacement method. In some embodiments, a prosthetic heart valve can be configured into a low-profile configuration for containment within a small diameter delivery sheath. For example, in some embodiments, a delivery sheath having an outer diameter of about a 10 to 12 French size is used to deploy a prosthetic aortic valve provided herein. Such a low-profile is achievable because while in the low-profile configuration the leaflets of the prosthetic aortic valve are partially detached from the valve stent (the prosthetic valve annulus). During the deployment process, as the prosthetic aortic valve is caused to emerge from the delivery sheath, the valve leaflets are assembled with the valve stent in situ.

While the inventive concepts provided herein are primarily described in the context of a prosthetic aortic valve, other applications of the concepts are also envisioned and within the scope of this disclosure. For example, the inventive concepts can be applied in the context of other heart valves such as, but not limited to, a prosthetic mitral valve. Further, in another implementation the inventive concepts provided herein can be applied in the context of an electro-anatomical mapping and/or ablation device. For example, the spiral leaflets can be embeded with electrodes and the stent portion removed. Such a structure can allow the unfurled leaflets to have wide surface area in contact within various structures such as the left ventricle, pulmonary artery, real arteries, or esophagus for purposes of electroanatomical mapping and ablation. Advantageously, this embodiment provides for lumen patency during contact, improving the efficacy of any ablation by preserving blood flow.

In general, one aspect of this document features a prosthetic aortic valve device. The prosthetic aortic valve device comprises a stent portion and a plurality of leaflets. The stent portion comprises a plurality of elongate members that form an open cylinder defining an interior space. The cylinder has a distal end and a proximal end. The stent portion is configurable between a collapsed configuration and an expanded configuration. The stent portion is configured to self-expand from the collapsed configuration to the expanded configuration when the stent portion is cause to emerge from containment within a delivery sheath. The plurality of leaflets are comprised of a flexible material. The leaflets have a partially disassembled delivery configuration and an assembled configuration. In the partially disassembled delivery configuration the leaflets are at least partially located outside of the interior space.

In various implementations, in the assembled configuration the leaflets may be joined together in a generally cylindrical shape, and the leaflets may be located within the interior space when the stent portion is in the expanded configuration. The leaflets may be attached to each other at struts that are attached to the stent portion when the leaflets are in the assembled configuration and the stent portion is in the expanded configuration. The leaflets may include a reinforcing member located along one edge of the leaflets, and the reinforcing member may be attached to the stent portion when the leaflets are in the assembled configuration and the stent portion is in the expanded configuration.

In another general aspect, this document features a TAVR system. The TAVR system comprises an elongate delivery sheath having an interior lumen; a catheter that is disposable within the lumen; a prosthetic aortic valve device; and a balloon that has an inflated configuration and a deflated configuration in which the balloon can be contained within the lumen. The prosthetic aortic valve device comprises: a stent portion that is reconfigurable between a collapsed configuration for containment within the lumen and an expanded configuration; and a plurality of leaflets comprising a flexible material. The leaflets have a partially disassembled delivery configuration in which the leaflets are spiraled around the catheter and an assembled configuration. In the partially disassembled delivery configuration the leaflets are at least partially located outside of the interior space. The stent portion is configured to self-expand from the collapsed configuration to the expanded configuration when the stent portion is cause to emerge from containment within the delivery sheath.

In various implementations, the TAVR method may include that in the assembled configuration the leaflets are joined together in a generally cylindrical shape, and the leaflets are located within an interior space defined by the stent portion when the stent portion is in the expanded configuration. The leaflets may be attached to each other at struts that are attached to the stent portion when the leaflets are in the assembled configuration and the stent portion is in the expanded configuration. The leaflets may include a reinforcing member located along one edge of the leaflets, and the reinforcing member may be attached to stent portion when the leaflets are in the assembled configuration and the stent portion is in the expanded configuration.

In another general aspect, this document features a TAVR method. The TAVR method comprises installing the following components within a lumen of a delivery sheath: a catheter; a balloon that has an inflated configuration and a deflated configuration in which the balloon can be contained within the lumen; and a prosthetic aortic valve device. The prosthetic aortic valve device comprises: a stent portion that is configurable between a collapsed configuration for containment within the lumen and an expanded configuration, wherein the stent portion is configured to self-expand from the collapsed configuration to the expanded configuration when the stent portion is cause to emerge from containment within the delivery sheath; and a plurality of leaflets comprising a flexible material, wherein the leaflets have a partially disassembled delivery configuration in which the leaflets are spiraled around the catheter and an assembled configuration, and wherein in the partially disassembled delivery configuration the leaflets are at least partially located outside of the interior space. The TAVR method further comprises: percutaneously inserting the delivery sheath within a patient's vasculature and navigating a distal tip of the delivery sheath to a location near the patient's aortic valve; causing the stent portion to emerge from the distal tip of the delivery sheath such that the stent portion self-expands to the expanded configuration within the patient's aortic valve; causing the leaflets to reconfigure from the disassembled delivery configuration to the assembled configuration; and inflating the balloon to force the leaflets to attached to the stent portion.

Particular embodiments of the subject matter described in this document can be implemented to realize one or more of the following advantages. In some embodiments, heart conditions such as valvular stenosis can be treated using the devices and methods provided herein. Some patients who would be too high risk for a traditional surgical valve replacement procedure can be treated using the prosthetic valve devices and transcatheter heart valve replacement methods provided herein. In some embodiments, the prosthetic aortic valves provided herein can be deployed within a patient using a delivery sheath with a small outer diameter of about a 10 to 12 French size. In some circumstances, using such a small diameter delivery sheath can reduce the risks of post procedure complications that may be associated with some current TAVR procedures.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1A is a schematic diagram of a human heart shown in partial cross-section undergoing a catheterization using a delivery sheath that is used to deploy a prosthetic aortic valve device for a TAVR procedure in accordance with some embodiments provided herein.

FIG. 1B is a schematic diagram of the human heart of FIG. 1A showing the prosthetic aortic valve device after being implanted using a TAVR procedure in accordance with some embodiments provided herein.

FIG. 2A illustrates a fully assembled prosthetic aortic valve device that can be implanted using a TAVR procedure in accordance with some embodiments provided herein.

FIG. 2B illustrates the prosthetic aortic valve device of FIG. 2A with the valve leaflets in a closed orientation.

FIG. 3A illustrates a TAVR system that includes a delivery sheath (shown in cross-section) containing a partially disassembled prosthetic aortic valve device in a low-profile delivery configuration in accordance with some embodiments provided herein.

FIG. 3B illustrates the system of FIG. 3A with a stent portion of the partially disassembled prosthetic aortic valve device being emerged from the delivery sheath and enlarged to an expanded configuration, while portions of the valve leaflets remain within the delivery sheath.

FIG. 3C illustrates the system of FIG. 3A with the valve leaflets of the partially disassembled prosthetic aortic valve device having emerged from the delivery sheath and being positioned within the stent portion of the valve device in preparation for being assembled in situ.

FIG. 3D illustrates the system of FIG. 3A with the valve leaflets of the prosthetic aortic valve device being assembled within the stent portion of the valve device using a crimping balloon in an expanded configuration.

FIG. 3E illustrates an end view of the configuration of FIG. 3D.

FIG. 4 is flowchart of a TAVR method in accordance with some embodiments provided herein.

Like reference numbers represent corresponding parts throughout.

### DETAILED DESCRIPTION

This document provides devices and methods for the treatment of heart conditions. For example, this document provides a prosthetic heart valve and a transcatheter heart valve replacement method. In some embodiments, a prosthetic heart valve can be configured into a low-profile delivery configuration for containment within a small diameter delivery sheath. For example, in some embodiments, a delivery sheath having an outer diameter of about a 10 to 12 French size is used to deploy the prosthetic aortic valve provided herein. Such a low-profile is achievable because while in the low-profile configuration the leaflets of the prosthetic aortic valve are partially detached from the valve stent (the prosthetic valve annulus). During the deployment process as the prosthetic aortic valve is caused to emerge from the delivery sheath, the valve leaflets become assembled with the valve stent in situ.

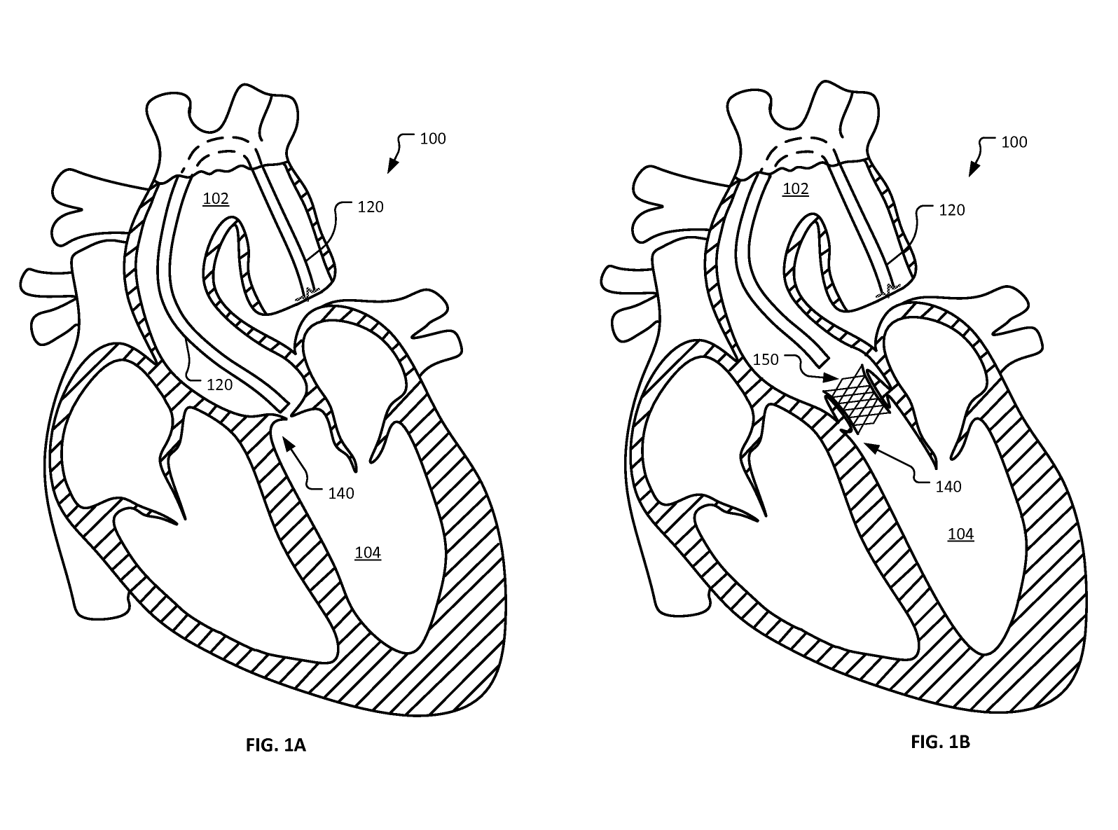

With reference to FIG. 1A, a schematic diagram is provided of human heart 100 shown in partial cross-section undergoing a catheterization of aortic arch 102 using a delivery sheath 120. Delivery sheath 120 is in aortic arch 102 for the purpose of transmitting a prosthetic aortic valve (not visible because the prosthetic aortic valve is within delivery sheath 120) to be implanted within an aortic valve 140.

In some cases, delivery sheath 120 can be percutaneously inserted in a femoral artery of a patient, and navigated to the patient's aorta 102 using imaging techniques such as fluoroscopy, MRI, or ultrasound. In some circumstances, a guidewire may be installed first. Radiopaque and/or echogenic markers can be included on delivery sheath 120 for enhanced imaging. From the aorta, delivery sheath 120 can be directed to aortic arch 102. In other cases, aortic arch 102 can be accessed by delivery sheath 120 via the patient's radial artery. Other aortic arch 102 access techniques are also envisioned, such as a transapical approach or a transvenous transeptal approach.

Other access techniques, including both retrograde and antegrade access options, are envisioned within the scope of this disclosure. Accordingly, as described further below, the prosthetic heart valves provided herein can be configured differently depending on whether the valve will be deployed using a retrograde or an antegrade approach.

With reference to FIG. 1B, a prosthetic aortic valve 150 is shown after being deployed from delivery sheath 120 and implanted within aortic valve 140. The valve stent portion of prosthetic aortic valve 150 is visible. The stent portion is an open cylinder that conforms to the anatomy of the patient at the implant site. Prosthetic aortic valve 150 hereafter takes over the function of the patient's natural aortic valve 140. Delivery sheath 120 can then be removed from the patient. This depiction of the delivery and implantation of prosthetic aortic valve 150 using delivery sheath 120 illustrates a TAVR procedure that will be described in more detail below.

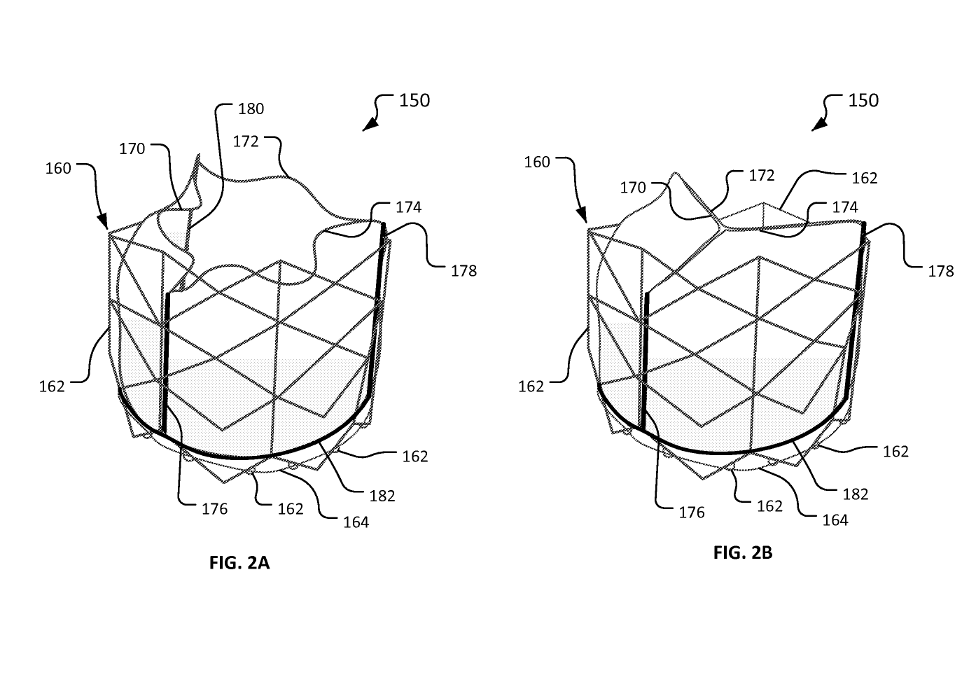

Referring to FIGS. 2A and 2B, a fully assembled prosthetic aortic valve 150 can include a valve stent portion 160 and multiple leaflets, such as three leaflets 170, 172, and 174. FIG. 2A shows prosthetic aortic valve 150 with leaflets 170, 172, and 174 in an open arrangement whereby blood can flow through prosthetic aortic valve 1510. FIG. 2B shows prosthetic aortic valve 150 with leaflets 170, 172, and 174 in a closed arrangement whereby blood is blocked from flowing (at least blocked from flowing from the aortic arch to the left ventricle). As will be described in more detail, prosthetic aortic valve 150 has an assembled configuration as shown, and a low-profile partially disassembled configuration that prosthetic aortic valve 150 is in while contained in a delivery sheath. Prosthetic aortic valve 150 is assembled in situ to arrive at the configuration shown.

Valve stent portion 160 is a generally cylindrical framework made up of multiple elongate frame members 162. In some embodiments, frame members 162 are a compilation of elongate wire members that are attached together to form a framework that creates the open cylindrical shape. Alternatively, frame members 162 can originally be a tube (e.g., a nitinol tube) that is laser cut and expanded into to the desired open cylindrical configuration, and heat-set to make the cylinder the natural configuration of frame members 162. Frame members 162 can be metallic, for example, constructed of nitinol (NiTi), stainless steel, titanium, or a combination of materials. Frame members 162 can be wires that are wound and attached together (e.g., welded or glued) to create the cylindrical configuration. The configuration of frame members 162 shown is merely exemplary. Other configurations of frame members 162, in terms of the numbers of frame members 162 and the arrangement thereof, are envisioned and within the scope of this disclosure.

In some embodiments, stent portion 160 includes a covering on all or on some areas of stent portion 160 (however, in FIGS. 2A and 2B no such covering is shown for simplicity of visualization). In particular embodiments, the covering can be made of materials including, but not limited to, Dacron, polyester fabrics, polyethylene terephthalate (PET), Teflon-based materials, Polytetrafluoroethylene (PTFE), expanded Polytetrafluoroethylene (ePTFE), polyurethanes, silicone, Bio A, copolymers, film or foil materials, or combinations of the foregoing materials and/or like materials. In some embodiments, the covering material has a material composition and configuration that inhibits or prevents tissue ingrowth to the covering material. In some embodiments, the covering material, or portions thereof, has a microporous structure that provides a tissue ingrowth scaffold for durable sealing and supplemental anchoring strength of prosthetic aortic valve 150.

Coverings can be attached to frame members 162 in a variety of suitable manners well known to those of ordinary skill in the art. For example, in some embodiments, coverings are sewn to frame members 162. In some embodiments, coverings are glued to frame members 162. In some embodiments, frame members 162 are sandwiched between two layers of coverings. In some embodiments, a combination of such attachment methods are used. These and all other variations of frame member types, material compositions, material treatments, configurations, fabrication techniques, and methods for attaching coverings to frame members 162 are envisioned and within the scope of the centering devices provided herein.

In general, valve stent portion 160 can be collapsed to a low-profile delivery configuration to fit within the lumen of a delivery sheath. In some embodiments, frame members 162 can radially self-expand to the cylindrical unconstrained configuration as shown when deployed from the delivery sheath. In particular embodiments, self-expanding frame members 162 can be comprised of super-elastic shape-memory nitinol (NiTi) material. In some embodiments, a secondary device such as a balloon is used to provide a temporary supplemental radial force to help expand frame members 162 into the cylindrical shape shown.

In some embodiments, frame members 162 include one or more visualization markers, such as radiopaque or echogenic markers, bands, or radiopaque filler materials. The radiopaque or echogenic markers can assist clinician (such as an interventional cardiologist) with in situ radiographic visualization of prosthetic aortic valve 150 so that the clinician can orient the device as desired in relation to the anatomy of the patient.

The end of stent portion 160 can include multiple eyelets 162 through which a retrieval cord 164 is threaded. While, in this embodiment, the distal end of the stent portion 160 has eyelets 162, in other embodiments the opposite end (the proximal end) or both ends can have eyelets 162. Eyelets 162 and retrieval cord 164 are used to retrieve or reposition prosthetic aortic valve 150. For example, a grasping device (not shown) can be routed to the site of prosthetic aortic valve 150 (such as through the delivery sheath or independently), and the grasping device can be used to attach onto retrieval cord 164. The grasping device can be used to pull on retrieval cord 164, which causes eyelets 162 to collapse toward each other like a purse when a purse string is used to cinch the purse closed. In the collapsed configuration, prosthetic aortic valve 150 can be repositioned or retrieved into a sheath for removal from the patient's body. In some embodiments, retrieval cord 164 remains coupled to stent portion 160 when prosthetic aortic valve 150 is in use in an aortic valve of a patient. Retrieval cord 164 can be made of polymer materials such as, but not limited to, nylon, polypropylene, PTFE, silk, and the like. In some embodiments, retrieval cord 164 can be a wire made of a metallic material including, but not limited to, nitinol, aluminum, stainless steel, and the like. Retrieval cord 164 can be a monofilament or braided and the like.

Leaflets 170, 172, and 174 can be made of flexible biological tissue materials or flexible synthetic materials. For example, in some embodiments leaflets 170, 172, and 174 are made of bovine or equine pericardial tissue. In some embodiments, leaflets 170, 172, and 174 are made using a tissue printing process by which leaflets 170, 172, and 174 are made using the patient's own tissue. Alternatively, leaflets 170, 172, and 174 can be made of synthetic materials such as non-fabric fluorocarbon polymer flexible sheeting, knitted Teflon fabric (coated or uncoated), isotropic silicone, flourosilicone rubbers, and the like.

Leaflets 170, 172, and 174 are joined to each other and to stent portion 160 at three struts 176, 178, and 180. In some embodiments, struts 176, 178, and 180 are seams at which leaflets 170, 172, and 174 are joined to each other, such as by suturing, using mechanical clips, sewing, using adhesives, bonding, a mechanical channel, and by combinations thereof. In some embodiments, struts 176, 178, and 180 are or become attached to stent portion 160 using attachment features including, but not limited to, mechanical clips, barbs, hooks, channels, and the like. Reinforcement members may be included in or on struts 176, 178, and 180 to provide additional stiffness and rigidity. Such reinforcement members may be made of metallic or polymeric materials, and may have the attachment features extending therefrom.

Strut 176 can be attached to stent portion 160 when prosthetic aortic valve 150 is in the partially disassembled configuration. Struts 178 and 180 can be detached from stent portion 160 when prosthetic aortic valve 150 is in the partially disassembled configuration. Struts 178 and 180 can include attachment features on their outer surfaces that can become attached to stent portion 160 when prosthetic aortic valve 150 is in the fully assembled configuration.

A reinforcing member 182 can be attached to the distal or lower edge of leaflets 170, 172, and 174. Reinforcing member 182 forms a basal ring for leaflets 170, 172, and 174. Reinforcing member 182 may be made of metallic or polymeric materials. For example, in some embodiments reinforcing member 182 is a shape-memory material, such as but not limited to nitinol, that has been made to naturally seek the circular shape as shown. In some embodiments, reinforcing member 182 may have attachment features extending therefrom. Such attachment features may include, but are not limited to, mechanical clips, barbs, hooks, and the like. The attachment features can couple reinforcing member 182 to stent portion 160, or to a covering that is attached to stent portion 160 in some embodiments.

The ends of leaflets 170, 172, and 174 that are opposite of reinforcing member 182 are the free ends of leaflets 170, 172, and 174. The free ends of leaflets 170, 172, and 174 can flexibly move whereby prosthetic aortic valve 150 can transition from the open configuration of FIG. 2A to the closed configuration of FIG. 2B.

While leaflets 170, 172, and 174 are shown having approximately the same axial length as stent portion 160, that is not required. In some embodiments, stent portion 160 is longer than leaflets 170, 172, and 174, such as 1 to 1.5 times as long, 1.5 to 2 times as long, 2 to 3 times as long, or longer. In such embodiments, additional frame members 162 can be added as desired to increase the axial length of stent portion 160 in comparison to the embodiment shown. In alternative embodiments, stent portion 160 is shorter than leaflets 170, 172, and 174.

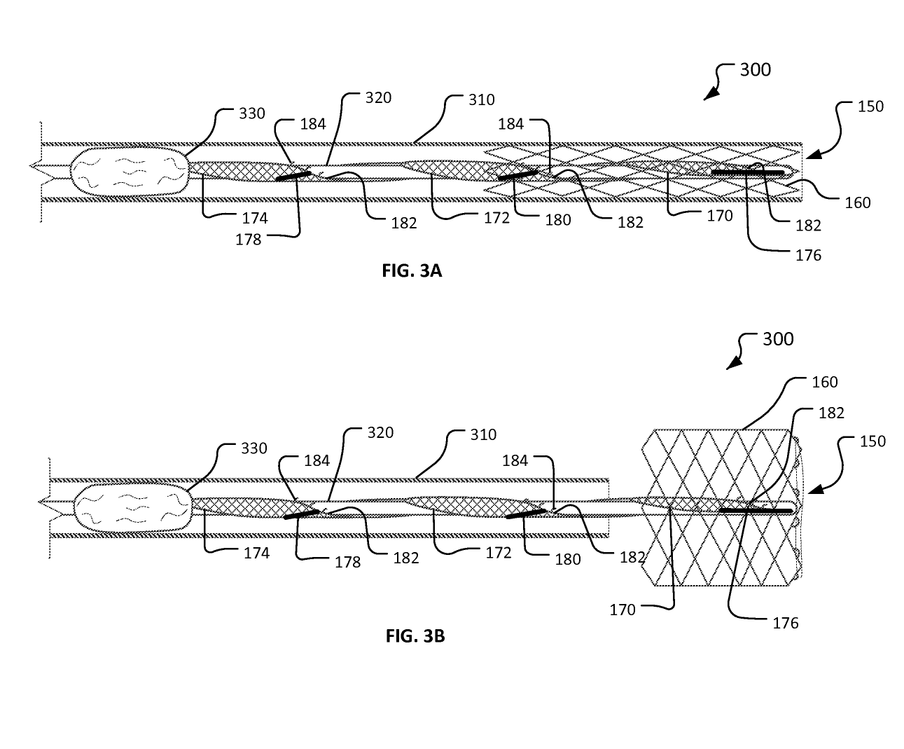

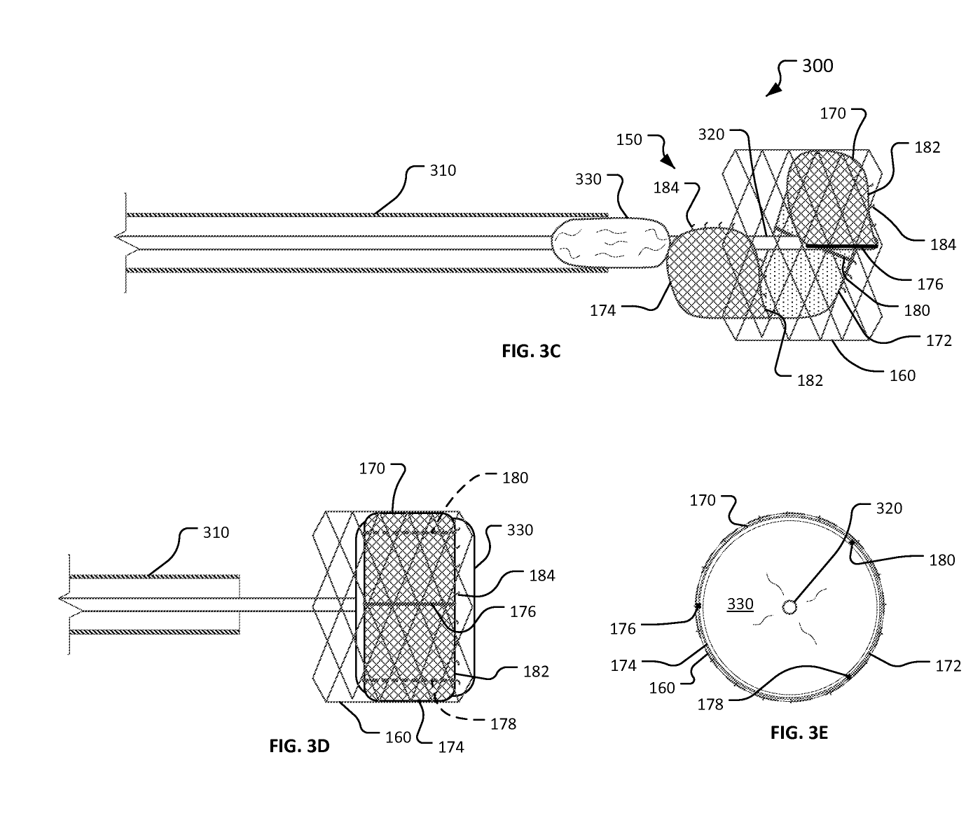

With reference to FIGS. 3A through 3E, a series of illustrations depicts a TAVR system 300 being used to deploy prosthetic aortic valve 150. TAVR system 300 includes, in addition to prosthetic aortic valve 150, a delivery sheath 310 with an interior lumen, a catheter 320, and a balloon 330. These figures illustrate how prosthetic aortic valve 150 can be reconfigured from a partially disassembled low-profile delivery configuration to a fully assembled configuration.

It should be understood that the embodiments depicted in FIGS. 3A through 3E are non-limiting. For example, while the depicted embodiment is configured in general for retrograde access, embodiments for antegrade access are also envisioned within the scope of this disclosure. That is, the design concepts disclosed herein can also be applied to create a prosthetic aortic valve that is configured to be delivered using an antegrade approach to the native valve site, as well as using a retrograde approach to the native valve site.

FIG. 3A illustrates prosthetic aortic valve 150 in the partially disassembled low-profile configuration within delivery sheath 310. Delivery sheath 360 is shown in cross-section so the contents within the lumen of delivery sheath 310 are visible. This is the arrangement in which delivery sheath 310 would be percutaneously inserted into a patient, and navigated within the vasculature of the patient to a position where the distal end of delivery sheath 310 is located near the patient's aortic valve (refer to FIG. 1A). In this configuration, balloon 330 is deflated.

In this partially disassembled low-profile configuration strut 176 is attached to valve stent portion 160, but leaflets 170, 172, and 174 are not otherwise attached to stent portion 160. Rather, leaflets 170, 172, and 174 are detached from stent portion 160 and are closely contained around catheter 320. In some embodiments, leaflets 170, 172, and 174 are wrapped around catheter 320. In some embodiments, leaflets 170, 172, and 174 are spirally-wrapped around catheter 320. Attachment features 184 are visible that will later be used to attach leaflets 170, 172, and 174 together and to stent portion 160. With leaflets 170, 172, and 174 disassembled in this arrangement, prosthetic aortic valve 150 can be collapsed to a smaller size than if prosthetic aortic valve 150 was fully assembled, i.e., with leaflets 170, 172, and 174 positioned within the interior space defined by stent portion 160. Accordingly, in some embodiments the outer diameter of delivery sheath 310 can be sized, for example, at about 8 to 10 French, about 10 to 12 French, or about 12 to 14 French.

The components of prosthetic aortic valve 150 will now be referred to so as to describe the partially-disassembled configuration of prosthetic aortic valve 150. Stent portion 160 is contained in a collapsed low-profile configuration within delivery sheath 310. Strut 176 is attached to stent portion 160. Leaflets 170, 172, and 174 are attached to stent portion 160 at strut 176 only. Otherwise, leaflets 170, 172, and 174 are detached from stent portion 160 and spiraled around catheter 320. Reinforcing member 182 is on one edge of spiraled leaflets 170, 172, and 174 and is spiraled around catheter 320 rather than being arranged circularly as in the assembled configuration. Struts 178 and 180 are detached from stent portion 160. Struts 178 and 180 are at the interfaces of leaflets 170, 172, and 174 on catheter 320. In some embodiments, a low-tack biocompatible adhesive is used to temporarily secure leaflets 170, 172, and 174 to catheter 320. In some embodiments, leaflets 170, 172, and 174 are merely maintained in tension to hold them in close contact with catheter 320.

FIG. 3B illustrates the beginning of the deployment process of prosthetic aortic valve 150 from delivery sheath 310 with the emergence of valve stent portion 160. This configuration can be the result, for example, of pulling back delivery sheath 310 while holding catheter 320 substantially stationary. Alternatively, catheter 320 can be pushed distally while holding delivery sheath 310 substantially stationary. As stent portion 160 emerges from containment within delivery sheath 310, stent portion 160 can self-expand within the aortic valve at the patient's aortic root (refer to FIG. 1B).

FIG. 3C illustrates the unwrapping of leaflets 170, 172, and 174 from their spiraled configuration on catheter 320. In some embodiments, balloon 330 can slide distally in relation to catheter 320 so as to force leaflets 170, 172, and 174 off of catheter 320 and into the interior space defined by stent portion 160. In alternative embodiments, balloon 330 is stationary in relation to catheter 320, and catheter 320 can be moved distally in relation to stent portion 160 so as to move leaflets 170, 172, and 174 off of catheter 320 and into the interior space defined by stent portion 160.

As leaflets 170, 172, and 174 become unwrapped from catheter 320, reinforcing member 182, having a shape memory, can naturally seek a circular shape within the distal end of the interior space defined by stent portion 160. At the end of this phase, leaflets 170, 172, and 174 are arranged in a cylindrical shape within the interior space defined by stent portion 160. Attachment features 184 are positioned to attached leaflets 170, 172, and 174 together and to stent portion 160, but attachment features 184 may not be fully engaged at this point. The attachment features 184 are therefore selectively attachable.

FIGS. 3D and 3E illustrate balloon 330 being used in an inflated configuration whereby attachment features 184 are forced into engagement with stent portion 160 and strut 176 to complete the assembly of prosthetic aortic valve 150. To accomplish this, balloon 330, while still deflated, is positioned within the interior space defined by stent portion 160 and leaflets 170, 172, and 174. Then balloon 330 is inflated in a known manner. Balloon 330 exerts radial forces on leaflets 170, 172, and 174 including on engagement features 184. Accordingly, engagement features 184 become coupled as designed. That is, engagement features 184 near the distal end of the leaflets 170, 172, and 174 (such as on reinforcing member 182) become coupled with stent portion 160 and/or with covering material disposed on stent portion 160. In addition, engagement features 184 on the end of leaflet 174 become coupled with strut 176, whereby leaflets 170, 172, and 174 form a cylindrical shape. Further, attachment features on struts 178 and 180 become coupled with stent portion 160. Afterwards, balloon 330 can be deflated and withdrawn into delivery sheath 310. Delivery sheath 310, containing catheter 320 and balloon 330 can then be removed from the patient.

While in the depicted embodiment, attachment features 184 are fixed to leaflets 170, 172, and 174 and attachment features 184 are selectively attachable to stent portion 160, in some embodiments the reverse is true. That is, in some embodiments attachment features 184 are fixed to stent portion 160 and attachment features 184 are selectively attachable to leaflets 170, 172, and 174. Alternatively, both stent portion 160 and leaflets 170, 172, and 174 may have attachment features 184 that are fixed thereto and selectively attachable with the other.

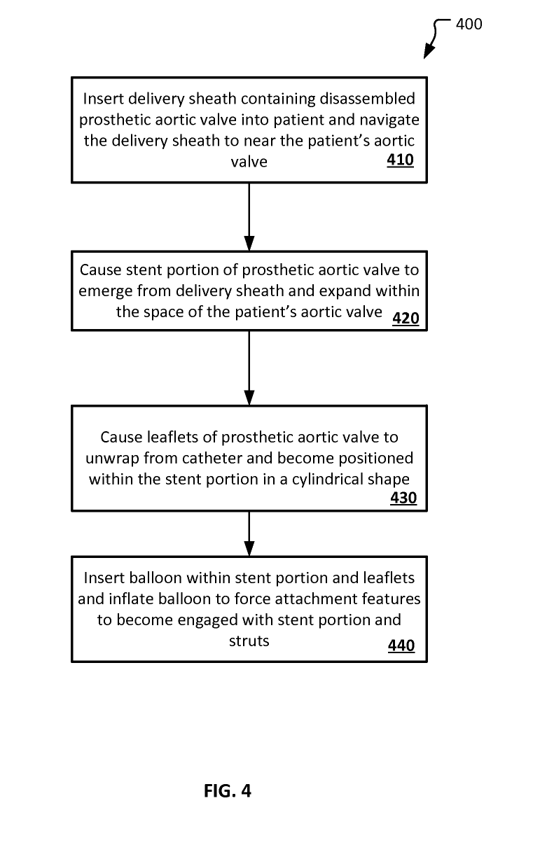

FIG. 4 provides a flowchart that describes a TAVR method 400. At step 410, a TAVR system including a delivery sheath containing a partially disassembled prosthetic aortic valve, a catheter, and a deflated balloon is inserted into a patient. The delivery sheath is navigated through the vasculature of the patient to the area of the patient's aortic valve.

At step 420, the delivery sheath is pulled back in relation to the catheter so that the valve stent portion of the partially disassembled prosthetic aortic valve emerges from the distal tip of the delivery sheath. The stent portion self-expands within the space of the patient's aortic valve.

At step 430, the leaflets of the partially disassembled prosthetic aortic valve, which had been spiraled around the catheter, are caused to unwrap from the catheter and become positioned within the stent portion in a generally cylindrical shape.

At step 440, the balloon is inserted to within the space of the stent portion and leaflets. The balloon is then inflated to force the leaflets to become attached to the stent portion and to each other. Afterwards, the balloon is deflated and withdrawn into the delivery sheath. The delivery sheath containing the balloon and catheter can then be removed from the patient, while the fully assembled prosthetic aortic valve remains implanted within the space of the patient's aortic valve.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A prosthetic aortic valve device comprising:
a stent portion comprising a plurality of elongate members that form an open cylinder defining an interior space and having a distal end and a proximal end, wherein the stent portion is configurable between a collapsed configuration and an expanded configuration, and wherein the stent portion is configured to self-expand from the collapsed configuration to the expanded configuration when the stent portion is cause to emerge from containment within a delivery sheath; and
a plurality of leaflets comprising a flexible material, the leaflets having a partially disassembled delivery configuration and an assembled configuration, wherein in the partially disassembled delivery configuration the leaflets are at least partially located outside of the interior space.

2. The prosthetic aortic valve device of claim 1, wherein in the assembled configuration the leaflets are joined together in a generally cylindrical shape, and wherein the leaflets are located within the interior space when the stent portion is in the expanded configuration.

3. The prosthetic aortic valve device of claim 1, wherein the leaflets are attached to each other at struts that are attached to the stent portion when the leaflets are in the assembled configuration and the stent portion is in the expanded configuration.

4. The prosthetic aortic valve device of claim 1, wherein the leaflets include one or more reinforcing members located along one edge of the leaflets, and wherein each of the one or more reinforcing members is attached to the stent portion when the leaflets are in the assembled configuration and the stent portion is in the expanded configuration.

5. The prosthetic aortic valve device of claim 4, wherein the one or more reinforcing members comprises three reinforcing members.

6. The prosthetic aortic valve device of claim 1, further comprising a plurality of attachment features fixed to the plurality of leaflets, wherein the plurality of attachment features are selectively attachable to the stent portion.

7. The prosthetic aortic valve device of claim 1, further comprising a plurality of attachment features fixed to the stent portion, wherein the plurality of attachment features are selectively attachable to the plurality of leaflets.

8. A TAVR system comprising:
an elongate delivery sheath having an interior lumen;
a catheter that is disposable within the lumen;
a prosthetic aortic valve device comprising:
a stent portion that is configurable between a collapsed configuration for containment within the lumen and an expanded configuration, and wherein the stent portion is configured to self-expand from the collapsed configuration to the expanded configuration when the stent portion is cause to emerge from containment within the delivery sheath; and
a plurality of leaflets comprising a flexible material, the leaflets having a partially disassembled delivery configuration in which the leaflets are spiraled around the catheter and an assembled configuration, wherein in the partially disassembled delivery reconfiguration the leaflets are at least partially located outside of the interior space; and
a balloon that has an inflated configuration, and wherein the balloon has a deflated configuration in which the balloon can be contained within the lumen.

9. The TAVR system of claim 8, wherein in the assembled configuration the leaflets are joined together in a generally cylindrical shape, and wherein the leaflets are located within an interior space defined by the stent portion when the stent portion is in the expanded configuration.

10. The TAVR system of claim 8, wherein the leaflets are attached to each other at struts that are attached to the stent portion when the leaflets are in the assembled configuration and the stent portion is in the expanded configuration.

11. The TAVR system of any of claim 8, wherein the leaflets include one or more reinforcing members located along one edge of the leaflets, and wherein each of the one or more reinforcing members is attached to stent portion when the leaflets are in the assembled configuration and the stent portion is in the expanded configuration.

12. The TAVR system of claim 11, wherein the one or more reinforcing members comprises three reinforcing members.

13. The TAVR system of any of claim 8, further comprising a plurality of attachment features fixed to the plurality of leaflets, wherein the plurality of attachment features are selectively attachable to the stent portion.

14. The TAVR system of any of claim 8, further comprising a plurality of attachment features fixed to the stent portion, wherein the plurality of attachment features are selectively attachable to the plurality of leaflets.

15. A TAVR method comprising:
installing within a lumen of a delivery sheath:
a catheter;
a prosthetic aortic valve device comprising:
a stent portion that is configurable between a collapsed configuration for containment within the lumen and an expanded configuration, and wherein the stent portion is configured to self-expand from the collapsed configuration to the expanded configuration when the stent portion is cause to emerge from containment within the delivery sheath; and
a plurality of leaflets comprising a flexible material, the leaflets having a partially disassembled delivery configuration in which the leaflets are closely contained around the catheter and an assembled configuration, wherein in the partially disassembled delivery configuration the leaflets are at least partially located outside of the interior space; and
a balloon that has an inflated configuration and a deflated configuration in which the balloon can be contained within the lumen;
percutaneously inserting the delivery sheath within a patient's vasculature and navigating a distal tip of the delivery sheath to a location near the patient's aortic valve;
causing the stent portion to emerge from the distal tip of the delivery sheath such that the stent portion self-expands to the expanded configuration within the patient's aortic valve;
causing the leaflets to reconfigure from the disassembled delivery configuration to the assembled configuration; and
inflating the balloon to force the leaflets to attach to the stent portion.

16. The TAVR method of claim 15, further comprising a plurality of attachment features fixed to the plurality of leaflets, wherein the inflating the balloon to force the leaflets to attach to the stent portion comprises attachment of at least some of the plurality of attachment features to the stent portion.

17. The TAVR method of claim 15, further comprising a plurality of attachment features fixed to the stent portion, wherein the inflating the balloon to force the leaflets to attach to the stent portion comprises attachment of at least some of the plurality of attachment features to the plurality of leaflets.

18. The TAVR method of claim 15, wherein the leaflets in the partially disassembled delivery configuration are wrapped around the catheter.

19. The TAVR method of claim 15, wherein the leaflets in the partially disassembled delivery configuration are spirally-wrapped around the catheter.
